(19) United States
(12) Patent Application Publication
(10) Pub. No.: US 2005/0180939 A1
(43) Pub. Date: Aug. 18, 2005

(54) Skin cleansing composition

(75) Inventor: Angeles Fonolla Moreno, Paris (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'Oreal, Paris (FR)

(21) Appl. No.: 11/033,881

(22) Filed: Jan. 13, 2005

Related U.S. Application Data
(60) Provisional application No. 60/540,327, filed on Feb. 2, 2004.

(30) Foreign Application Priority Data
Jan. 27, 2004 (FR) 0450137

(51) Int. Cl. A61K 7/06; A61K 7/11; A61K 7/075; A61K 7/08

(52) U.S. Cl. 424/70.13; 424/70.14; 424/70.23; 424/70.24

(57) ABSTRACT

A composition containing, in a physiologically acceptable aqueous medium, (1) a surfactant system containing at least one anionic surfactant and at least one amphoteric surfactant, and (2) a thickening system containing at least two different anionic polymers each comprising at least one hydrophobic chain.

### REFERENCE TO PRIOR APPLICATIONS

This application claims priority to U.S. provisional application 60/540,327 filed Feb. 2, 2004, and to French patent application 0450137 filed Jan. 27, 2004, both incorporated herein by reference.

### FIELD OF THE INVENTION

The present invention relates to a cleansing composition comprising a particular surfactant system and a particular thickening system, and to the use of the composition, in particular in the cosmetic field as cleansing or make-up-removing products for the skin, including the eyelids, and as cleansing products for the hair. Preferably, the composition is rinsable and foaming.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

Cleansing the skin is very important for facial care. It must be as efficient as possible because greasy residues such as excess sebum, the remnants of cosmetic products used daily and make-up products accumulate in the skin folds and can block the pores of the skin and cause the appearance of spots.

One means of cleansing the skin properly is to use foaming cleansing products. The foaming cleansing products which are currently commercially available are in the form of foaming cakes, gels or creams and may or may not comprise soaps (fatty acid salts). Foaming products with soaps have the advantage of giving an unctuous foam; however, some consumers criticize these products for causing tightness due to their excessively high detergency. To have a product which is better tolerated, it is sought to reduce the soap content. However, the product then has insufficient viscosity and foaming power.

Moreover, soap-free foaming products are generally well tolerated. They are most often in the form of liquid products or gels. These products are used like the products with soap, that is to say that the product is applied to the skin and moistened with water in order to emulsify the product on the skin, then rinsed with water. It is known in particular to use foaming aqueous gels which are easier to handle than liquid products because they do not run. Their cleansing action is provided by the surfactants which they comprise, these surfactants suspending the greasy residues and the pigments of the make-up products. It is sought in particular to make transparent foaming cleansing gels because, like water, transparency is the symbol of purity and therefore of cleanliness, and transparent gels are thus particularly appreciated by users.

However, the presence of thickeners in the gel often affects its foam qualities and induces in particular poor initiation of the foam. Furthermore, after rinsing, the skin is often slippery and there is no feeling of a clean skin because of the presence of a film-forming residue on the skin which is difficult to remove.

Thus, the need remains for soap-free foaming cleansing products which are provided in the form of gels, in particular transparent or translucent gels, which have the properties required for foaming products, namely good mixing with water, rapid conversion to foam and good rinsing, while having the desired viscosity.

The Applicant has discovered, surprisingly, that the use of a particular surfactant system and of a particular thickening system made it possible to obtain foaming products, which run under their own weight while being fairly thick, that is to say having the viscosity of a gel, and having good foaming properties, that is to say easy and rapid initiation of foam and good rinsing power during use. These products are unctuous and have good cosmetic properties.

### DETAILED DESCRIPTION OF THE PREFERRED EMBODIMENTS

One subject of the present application is a cleansing composition comprising in a physiologically acceptable aqueous medium (1) a surfactant system comprising at least one anionic surfactant and at least one amphoteric surfactant, and (2) a thickening system comprising at least two different anionic polymers each comprising at least one hydrophobic chain (the hydrophobic chains of the two anionic polymers may be the same or different, and each anionic polymer may contain more than one type of hydrophobic chain). The composition may constitute a cosmetic or dermatological composition. It is preferably a cosmetic composition, is preferably rinsable, and preferably foaming.

The expression “physiologically acceptable medium” is understood to mean here a medium compatible with the skin, the mucous membranes, the scalp, the eyes and/or the hair. Moreover, this includes an aqueous medium, that is to say a medium comprising water. The quantity of water is preferably at least 35% by weight; it ranges preferably from 35 to 98% by weight and even better from 40 to 95% by weight relative to the total weight of the composition.

The compositions of the invention are preferably foaming and rinsable cleansing compositions. They are preferably in the form of a gel capable of running under its own weight without being liquid, that is to say having a viscosity which may range for example from 3 to 200 poises (0.3 to 20 Pa.s), and even better from 5 to 150 poises (0.5 to 15 Pa.s), and better still from 10 to 120 poises (1 to 12 Pa.s), measured at 25° C. with the aid of the Rheomat RM180 from Rheometric Scientific, this apparatus being equipped with a different rotor according to the viscosities, for example with a No. 2 rotor for viscosity ranges of less than 7 poises, and with a No. 3 rotor for viscosity ranges of 2 to 40 poises, and with a No. 4 rotor for viscosity ranges of 20 poises to 120 poises.

The compositions according to the invention preferably have a pH which is compatible with applications to the skin, generally of between 4.5 and 7.

The gel obtained is generally preferably transparent or translucent, which means that the composition has a turbidity of less than or equal to 500 NTU. NTUs (Nephelometric Turbidity Units) are the units of measurement of turbidity of a composition. The measurement of turbidity may be carried out for example with a turbidimeter model 2100P from the company HACH Compagny, the tubes used for the measurement being identified by the references AR397A cat 24347-06. The measurements are carried out at room temperature (20° C. to 25° C.). The transparency of a composition may be measured either by the coefficient of transmittance at 600 nm or by the turbidity. The composition of the invention has a coefficient of transmittance at 600 nm ranging from 10 to 90% or a turbidity ranging from 2 to 500 NTU, and preferably from 5 to 300 NTU.

Surfactant System

The composition according to the invention comprises a surfactant system which preferably will provide a foaming character to the composition and which comprises at least one anionic surfactant and at least one amphoteric surfactant. These surfactants are foaming surfactants. This system may optionally comprise one or more nonionic foaming surfactants.

The quantity of surfactant system, that is to say the total quantity of surfactants in the composition, may range for example, by weight (of active substance), from 0.5 to 40% by weight, preferably from 1 to 25% by weight and even better from 1 to 20% by weight relative to the total weight of the composition.

The anionic surfactants may be chosen in particular from the anionic derivatives of proteins of plant origin, amino acid derivatives, alkyl sulphates, alkyl ether sulphates, sulphonates, isethionates, taurates, sulphosuccinates, alkyl sulphoacetates, phosphates and alkyl phosphates, polypeptides, anionic derivatives of alkyl polyglucoside, and mixtures thereof.

The anionic derivatives of proteins of plant origin are preferably hydrolysates of protein comprising a hydrophobic group, it being possible for the hydrophobic group to be naturally present in the protein or to be added by reacting the protein and/or the protein hydrolysate with a hydrophobic compound. The proteins are of plant origin, and the hydrophobic group may be in particular a fatty chain, for example an alkyl chain comprising from 10 to 22 carbon atoms.

As anionic derivatives of proteins of plant origin which can be used in the composition according to the invention, there may be mentioned more particularly hydrolysates of wheat, soybean, oat or silk proteins comprising an alkyl chain having from 10 to 22 carbon atoms and their salts. The alkyl chain may be in particular a lauryl chain and the salt may be a sodium, potassium and/or ammonium salt. There may be mentioned for example sodium, potassium and/or ammonium salts of hydrolysates of silk protein modified with lauric acid, such as the product marketed under the name KAWA SILK by the company Kawaken; the sodium, potassium and/or ammonium salts of hydrolysates of wheat protein modified with lauric acid, such as the potassium salt marketed under the name AMINOFOAM W OR by the company Croda (CTFA name: Potassium lauroyl wheat amino acids) and the sodium salt marketed under the name PROTEOL LW 30 by the company Seppic (CTFA name: sodium lauroyl wheat amino acids); the sodium, potassium and/or ammonium salts of hydrolysates of oat protein comprising an alkyl chain having from 10 to 22 carbon atoms, and more especially the sodium, potassium and/or ammonium salts of the hydrolysates of oat protein modified with lauric acid, such as the sodium salt marketed under the name PROTEOL OAT by the company Seppic (CTFA name: Sodium lauroyl oat amino acids).

The amino acid derivatives may be chosen, for example, from sarcosinates and in particular acylsarcosinates, for instance the sodium lauroyl sarcosinate sold under the name SARKOSYL NL 97® by the company Ciba or sold under the name ORAMIX L 30® by the company Seppic, the sodium myristoyl sarcosinate sold under the name NIKKOL SARCOSINATE MN® by the company Nikkol or the sodium palmitoyl sarcosinate sold under the name NIKKOL SARCOSINATE PN® by the company Nikkol; alaninates, for instance the sodium N-lauroyl-N-methylamidopropionate sold under the name SODIUM NIKKOL ALANINATE LN 30® by the company Nikkol or sold under the name ALANONE ALE® by the company Kawaken, and the N-lauroyl-N-methylalanine triethanolamine sold under the name ALANONE ALTA® by the company Kawaken; N-acylglutamates, for instance the triethanolamine monococoylglutamate sold under the name ACYLGLUTAMATE CT-12® by the company Ajinomoto and the triethanolamine lauroylglutamate sold under the name ACYLGLUTAMATE LT-12® by the company Ajinomoto; aspartates, for instance the mixture of triethanolamine N-lauroyl aspartate and of triethanolamine N-myristoylaspartate, sold under the name ASPARACK® by the company Mitsubishi; citrates.

Alkyl ether sulphates which may be mentioned, for example, include the sodium lauryl ether sulphate (C12-14 70/30) (2.2 EO) sold under the names SIPON AOS 225® or TEXAPON N702 PATE® by the company Henkel, the ammonium lauryl ether sulphate (C12-14 70/30) (3 EO) sold under the name SIPON LEA 370® by the company Henkel, and the ammonium (C12-C14) alkyl ether (9 EO) sulphate sold under the name RHODAPEX AB/20® by the company Rhodia Chimie.

Sulphonates which may be mentioned, for example, include alpha-olefin sulphonates, for instance the sodium alpha-olefin sulphonate (C14-16) sold under the name BIO-TERGE AS-40® by the company Stepan, sold under the names WITCONATE AOS PROTEGE® and SULFRAMINE AOS PH 12® by the company Witco or sold under the name BIO-TERGE AS-40 CG® by the company Stepan, the sodium secondary olefin sulphonate sold under the name HOSTAPUR SAS 30® by the company Clariant; linear alkyl aryl sulphonates, for instance the sodium xylene sulphonate sold under the names MANROSOL SXS30®, MANROSOL SXS40® and MANROSOL SXS93® by the company Manro.

Isethionates which may be mentioned include acylisethionates, for instance sodium cocoylisethionate, such as the product sold under the name JORDAPON CI P® by the company Jordan.

Taurates which may be mentioned include the sodium salt of palm kernel oil methyltaurate sold under the name HOSTAPON CT PATE® by the company Clariant; N-acyl N-methyltaurates, for instance the sodium N-cocoyl N-methyltaurate sold under the name HOSTAPON LT-SF® by the company Clariant or sold under the name NIKKOL CMT-30-T® by the company Nikkol, and the sodium palmitoyl methyltaurate sold under the name NIKKOL PMT® by the company Nikkol.

Sulphosuccinates which may be mentioned, for example, include the oxyethylenated (3 EO) lauryl alcohol monosulphosuccinate (C12/C14 70/30) sold under the names SETACIN 103 SPECIAL®, REWOPOL SB-FA 30 K 4® by the company Witco, the disodium salt of a C12-C14 alkyl hemisulphosuccinate, sold under the name SETACIN F SPECIAL PASTE® by the company Zschimmer Schwarz, the oxyethylenated (2 EO) disodium oleamidosulphosuccinate sold under the name STANDAPOL SH 135® by the company Henkel, the oxyethylenated (5 EO) laurylamide monosulphosuccinate sold under the name LEBON A-5000® by the company Sanyo, the oxyethylenated (10 EO) disodium salt of lauryl citrate monosulphosuccinate sold under the name REWOPOL SB CS 50® by the company Witco, and the ricinoleic monoethanolamide monosulphosuccinate sold under the name REWODERM S 1333® by the company Witco.

Phosphates and alkyl phosphates which may be mentioned, for example, include monoalkyl phosphates and dialkyl phosphates, such as the lauryl monophosphate sold under the name MAP 20® by the company Kao Chemicals, the potassium salt of dodecylphosphoric acid, as a mixture of monoester and diester (mainly diester), sold under the name CRAFOL AP-31® by the company Cognis, the mixture of monoester and diester of octylphosphoric acid, sold under the name CRAFOL AP-20® by the company Cognis, the mixture of ethoxylated (7 mol of EO) phosphoric acid monoester and diester of 2-butyloctanol, sold under the name ISOFOL 12 7 EO-PHOSPHATE ESTER® by the company Condea, the potassium salt or triethanolamine salt of monoalkyl (C12-C13) phosphate sold under the references ARLATONE MAP 230K-40® and ARLATONE MAP 230T-60® by the company Uniqema, and the potassium lauryl phosphate sold under the name DERMALCARE MAP XC-99/09® by the company Rhodia Chimie.

The anionic derivatives of alkyl poly-glucosides may be, in particular, glyceryl ethers, carbonates, sulphosuccinates, tartrates and citrates obtained from alkyl polyglucosides. Mention may be made, for example, of the sodium salt of cocoylpolyglucoside (1,4) tartaric ester, sold under the name EUCAROL AGE-ET® by the company Cesalpinia, the disodium salt of cocoylpolyglucoside (1,4) sulphosuccinic ester, sold under the name ESSAI 512 MP® by the company Seppic, and the sodium salt of cocoylpolyglucoside (1,4) citric ester, sold under the name EUCAROL AGE-EC® by the company Cesalpinia.

According to a preferred embodiment of the invention, the surfactant system comprises, as anionic surfactant, at least one anionic derivative of proteins of plant origin. This anionic surfactant may be chosen in particular from the sodium, potassium and/or ammonium salts of hydrolysates of oat protein comprising an alkyl chain having from 10 to 22 carbon atoms, and more particularly the sodium, potassium and/or ammonium salts of hydrolysates of oat protein modified with lauric acid, such as the product marketed under the name PROTEOL OAT by the company Seppic (CTFA name: Sodium Lauroyl Oat Amino Acids) (CTFA name), it being understood that it may additionally comprise one or more other anionic surfactants.

The amphoteric surfactants (this term including the amphoteric and zwitterionic surfactants) uselfu herein include those that may be chosen for example from betaines, N-alkylamidobetaines and derivatives thereof, glycine derivatives, sultaines, alkyl polyaminocarboxylates and alkylamphoacetates, and mixtures thereof.

Betaines which may be mentioned, for example, include cocobetaine, for instance the product sold under the name DEHYTON AB-30® by the company Henkel, laurylbetaine, for instance the product sold under the name GENAGEN KB® by the company Clariant, oxyethylenated (10 EO) laurylbetaine, for instance the product sold under the name LAURYL ETHER (10 EO) BETAINE® by the company Shin Nihon Rica, and oxyethylenated (10 EO) stearylbetaine, for instance the product sold under the name STEARYL ETHER (10 EO) BETAINE® by the company Shin Nihon Rica.

Among the N-alkylamidobetaines and derivatives thereof which may be mentioned, for example, are the cocamidopropylbetaine sold under the name LEBON 2000 HG® by the company Sanyo, or sold under the name EMPIGEN BB® by the company Albright & Wilson, and the lauramidopropyl betaine sold under the name REWOTERIC AMB12P® by the company Witco.

Glycine derivatives which may be mentioned include sodium N-cocoylglycinate sold under the name AMILITE GCS-12® by the company Ajinomoto.

Sultaines which may be mentioned include the cocoylamidopropylhydroxysulphobetaine sold under the name CROSULTAINE C-50® by the company Croda.

Alkyl polyaminocarboxylates (APACs) which may be mentioned include the sodium cocoylpolyaminocarboxylate sold under the name AMPHOLAK 7 CX/C® and AMPHOLAK 7 CX® by the company Akzo Nobel, the sodium stearylpolyamidocarboxylate sold under the name AMPHOLAK 7 TX/C by the company Akzo Nobel and the sodium carboxymethyloleylpolypropylamine sold under the name AMPHOLAK XO7/C® by the company Akzo Nobel.

Alkylamphoacetates which may be mentioned, for example, include N-disodium N-cocoyl-N-carboxymethoxyethyl-N-carboxymethylethylenediamine (CTFA name: disodium cocamphodiacetate), for instance the product sold under the name MIRANOL C2M CONCENTRE NP® by the company Rhodia Chimie and N-sodium N-cocoyl-N-hydroxyethyl-N-carboxymethylethylenediamine (CTFA name: sodium cocamphoacetate).

According to a preferred embodiment of the invention, the surfactant system comprises, as amphoteric surfactant, at least one betaine, in particular cocobetaine, it being understood that it may additionally comprise one or more other amphoteric surfactants.

As indicated above, the surfactant system used in the composition of the invention may additionally comprise one or more nonionic surfactants. These nonionic surfactants may be chosen for example from alkyl polyglucosides (APG), maltose esters, polyglycerolated fatty alcohols, glucamine derivatives such as 2-ethylhexyloxycarbonyl-N-methylglucamine, and mixtures thereof.

As alkyl polyglucosides, use is preferably made of those comprising an alkyl group comprising from 6 to 30 carbon atoms, and preferably from 8 to 16 carbon atoms, and comprising a hydrophilic (glucoside) group preferably comprising 1.2 to 3 saccharide units. As alkyl polyglucosides, there may be mentioned, for example, decyl glucoside (C9/C11 alkyl polyglucoside (1.4)) such as the product marketed under the name MYDOL 10® by the company Kao Chemicals, the product marketed under the name PLANTAREN 2000 UP® by the company Henkel, and the product marketed under the name ORAMIX NS 10® by the company Seppic; caprylyl/capryl glucoside such as the product marketed under the name ORAMIX CG 110® by the company Seppic; lauryl glucoside such as the products marketed under the names PLANTAREN 1200 N® and PLANTACARE 1200® by the company Henkel; and coco-glucoside such as the product marketed under the name PLANTACARE 818/UP® by the company Henkel.

The maltose derivatives are, for example, those disclosed in document EP-A-566 438, such as O-octanoyl-6′-D-maltose or O-dodecanoyl-6′-D-maltose disclosed in document FR-2 739 556.

Among the polyglycerolated fatty alcohols which may be mentioned are polyglycerolated dodecanediol (3.5 mol of glycerol), this product being sold under the name CHIMEXANE NF® by the company Chimex.

According to a preferred embodiment of the invention, the nonionic surfactant optionally present is preferably chosen from alkyl polyglucosides.

When the composition comprises nonionic surfactants, the quantity of nonionic surfactants (as active substance) may range for example from 0.5 to 20% by weight and even better from 0.5 to 15% by weight relative to the total weight of the composition.

Thickening System

The thickening system comprises at least two anionic polymers comprising at least one hydrophobic chain, these anionic polymers being different from each other.

The hydrophobic chain(s) of the anionic polymer used according to the invention are in particular saturated or unsaturated, linear or branched hydrocarbon chains having from 12 to 30 carbon atoms, such as alkyl, arylalkyl, alkylaryl, alkylene; cycloaliphatic divalent groups such as in particular methylenedicyclohexyl and isophorone; or aromatic divalent groups such as phenylene.

The anionic polymers used in the present invention may be chosen in particular from copolymers of carboxylic acid with α,β-monoethylenic unsaturation, in particular of acrylic or methacrylic acid. The expression “copolymers” is understood to mean both the copolymers obtained from two types of monomers and those obtained from more than two types of monomers such as the terpolymers obtained from three types of monomers.

The anionic polymers preferably used in the invention are copolymers obtained by copolymerization of one or more monomers (a) chosen from carboxylic acids with α,β-ethylenic unsaturation or esters thereof, with a monomer (b) with ethylenic unsaturation comprising a hydrophobic group.

The carboxylic acid with α,β-monoethylenic unsaturation constituting the monomer (a) may be chosen from numerous acids and in particular from acrylic acid, methacrylic acid, crotonic acid, itaconic acid, maleic acid and esters thereof. This preferably includes acrylic acid or methacrylic acid, and esters thereof.

The monomer (b) comprising a hydrophobic group may be chosen in particular from acrylates, methacrylates or itaconates of an oxyethylenated (1 to 50 EO) C₁₂-C₃₀ fatty alcohol, such as steareth-20 methacrylate, oxyethylenated (25 EO) behenyl methacrylate, oxyethylenated (20 EO) monocetyl itaconate, oxyethylenated (20 EO) monostearyl itaconate, acrylate modified with polyoxyethylenated (25 EO) C12-C24 alcohols, and among acrylates or methacrylates of C₁₂-C₃₀ fatty acids such as decyl, lauryl, stearyl, behenyl or melissyl acrylates or methacrylates, and mixtures thereof.

As anionic polymers comprising at least one monomer of oxyethylenated C₁₂-C₃₀ fatty alcohol acrylates, methacrylates or itaconates, there may be mentioned in particular the acrylates/steareth-20 methacrylate copolymer as such or in the form of an aqueous dispersion, such as the product sold under the name Aculyn 22 by the company Rohm & Haas (CTFA name: Acrylates/Steareth-30 Methacrylate copolymer); the oxyethylenated (25 EO) (meth)acrylic acid/ethyl acrylate/behenyl methacrylate terpolymer such as the product in aqueous emulsion sold under the name Aculyn 28 by the company Rohm & Haas; the oxyethylenated (20 EO) acrylic acid/monocetyl itaconate copolymer such as the product in aqueous dispersion at 30% sold under the name Structure 3001 by the company National Starch; the oxyethylenated (20 EO) acrylic acid/monostearyl itaconic copolymer such as the product in aqueous dispersion at 30% sold under the name Structure 2001 by the company National Starch; the acrylates/acrylate copolymer modified with polyethylenated (25 EO) C12-C24 alcohols such as the latex comprising 30-32% of copolymer, sold under the name Synthalen W2000 by the company 3V SA.

As anionic polymers comprising at least one monomer of acrylates or methacrylates of C₁₂-C₃₀ fatty acid, there may be mentioned more particularly the polymers comprising a monomer of decyl, lauryl, stearyl, behenyl or melissyl acrylates or methacrylates, more particularly stearyl acrylates and methacrylates, and in particular the copolymers sold under the names PEMULEN or CARBOPOL by the company Noveon, such as the acrylate/C₁₀-C₃₀ alkyl acrylate copolymer, for instance the products PEMULEN TR1, PEMULEN TR2 or CARBOPOL 1382 (CTFA name: Acrylates/C10-30 Alkyl acrylate Crosspolymer).

According to a preferred embodiment of the invention, the anionic polymers are chosen from copolymers comprising at least one monomer of acrylates, methacrylates or itaconates of oxyethylenated C₁₂-C₃₀ fatty alcohol, the copolymers comprising at least one monomer of acrylates or methacrylates of C₁₂-C₃₀ fatty acid, and mixtures thereof (1). Preferably, the thickening system comprises at least one copolymer comprising at least one monomer of acrylates, methacrylates or itaconates of oxyethylenated C₁₂-C₃₀ fatty alcohol, in particular acrylates/steareth-20 methacrylate copolymer, and (2) at least one copolymer comprising at least one monomer of acrylates or methacrylates of C₁₂-C₃₀ fatty acid, in particular stearyl acrylates or methacrylates. Preferably, the thickening system comprises the acrylates/steareth-20 methacrylate copolymer and the acrylates/C₁₀-C₃₀ alkyl acrylates copolymer.

According to the invention, the thickening system may represent, as active substance, from 0.05 to 10% by weight, preferably from 0.1 to 5% by weight, even better from 0.5 to 5% by weight relative to the total weight of the composition. Preferably, each of the polymers is present in a quantity of 0.01 to 9.99% by weight, preferably of 0.1 to 4.5% by weight relative to the total weight of the composition.

The composition according to the invention may additionally comprise one or more amphoteric polymers, such as the acrylic acid/methacrylate/methacrylamidopropyltrimonium chloride terpolymers, for instance POLYQUATERNIUM.47 (CTFA name) and for example the products sold under the names MERQUAT 2001, MERQUAT 280, MERQUAT 295 and MERQUAT PLUS 3330 by the company CALGON, or one or more cationic polymers such as the dimethyldiallylammonium chloride and acrylamide copolymer (CTFA name: Polyquaternium 7) and for example the products sold under the names MERQUAT S, MERQUAT 2200 and MERQUAT 550 by the company CALGON or the product sold under the name SALCARE SC 10 by the company Ciba. When they are present, the quantity of amphoteric and/or cationic polymers may range for example, as active substance, from 0.01 to 5% by weight and preferably from 0.02 to 3% by weight relative to the total weight of the composition.

The aqueous medium for the composition according to the invention may comprise, in addition to water, one or more solvents chosen from lower alcohols comprising from 1 to 6 carbon atoms, such as ethanol; and polyols. As polyols, there may be mentioned glycerine; glycols such as butylene glycol, isoprene glycol, propylene glycol, polyethylene glycols such as PEG-8; sorbitol; sugars such as glucose, fructose, maltose, lactose, sucrose; and mixtures thereof. The quantity of solvent(s) in the composition of the invention may range for example from 0.1 to 20% by weight and preferably from 0.5 to 15% by weight and even better from 0.5 to 10% by weight relative to the total weight of the composition.

The compositions of the invention may comprise adjuvants customarily used in the cosmetic field, and in particular those used in cleansing products. As adjuvants, there may be mentioned for example perfumes, preservatives, sequestrants (EDTA), pigments, exfoliating fillers, soluble colorants, sunscreens, cosmetic or dermatological active agents such as moisturizers, for instance hyaluronic acid; ceramides; water-soluble or fat-soluble vitamins, for example vitamin C and its derivatives such as vitamin CG; antiseptics; antiseborrhoeic; antimicrobials such as benzoyl peroxide, salicylic acid, triclosan, azelaic acid; optical brighteners. The quantities of these various adjuvants are those conventionally used in the field considered, and are for example from 0.01 to 20% of the total weight of the composition. These adjuvants, and their concentrations, should be such that they do not modify the property sought for the composition of the invention.

The compositions according to the invention may constitute in particular cleansing or make-up-removing products for the skin (body, face including eyelids and eyelashes), and/or cleansing products for the hair.

Another subject of the invention is the cosmetic use of the composition as defined above, as cleansing and/or make-up-removing product for the skin and as cleansing product for the hair.

Another subject of the invention is a cosmetic method for cleansing the skin, wherein the composition of the invention is applied to the skin, in the presence of water, and in that the foam formed and the dirt residues are removed by rinsing with water.

The examples which follow serve to illustrate the invention without however being of a limiting nature. The quantities indicated are in % by weight unless otherwise stated and correspond to the quantity of raw material and not to the quantity of active substance. The names of the raw materials are most often indicated as CTFA name.

### EXAMPLES 1 to 4 ACCORDING TO THE INVENTION AND COMPARATIVE EXAMPLES 5

| | Example 1 | Example 2 | Example 3 | | |
|---|---|---|---|---|---|
| | according | according | according | Example 4 | |
| | to the | to the | to the | according to | Comparative |
| Compositions | invention | invention | invention | the invention | Example 5 |
| Water | qs 100 | qs 100 | qs 100 | qs 100 | qs 100 |
| Acrylates/Steareth-30 | 1 | 2.5 | 0.5 | 3.5 | 1 |
| Methacrylate copolymer (1) | | | | | |
| Acrylates/C10-30 Alkyl | 0.3 | 0.4 | 0.5 | 0.4 | 0.3 |
| acrylate Crosspolymer (2) | | | | | |
| Triethanolamine | 0.5 | 0.65 | 0.7 | 0.6 | 0.5 |
| Cocobetaine (3) | 3 | 3 | 5 | 3.5 | 3 |
| Sodium Lauroyl Oat | 7 | 1.5 | 2 | 3 | 0 |
| Amino Acids (4) | | | | | |
| Vitamin CG (5) | 0.5 | 0.2 | 0.5 | 0.2 | 0.5 |
| Ammonium Lauryl | 11 | 10 | 9 | 8.5 | 11 |
| sulphate (6) | | | | | |
| TEA Lauryl Sulphate (7) | 2.5 | 2 | 3 | 4 | 2.5 |
| Cocoglucoside (8) | 2.5 | 1.5 | 1.5 | 3 | 2.5 |
| Decyl glucoside (9) | 1.5 | 2 | 1.5 | 2.5 | 1.5 |
| Glycerine | 4 | 3 | 4 | 2.5 | 4 |
| Polyquaternium 47 (10) | 0.5 | 0.5 | 0 | 0.3 | 0.5 |
| Disodium EDTA | 0.2 | 0.2 | 0.2 | 0.2 | 0.2 |
| Mint extract (11) | 0.3 | 0.4 | 0.6 | 0.5 | 0.3 |
| Perfume | 0.3 | 0.3 | 0.3 | 0.3 | 0.3 |
| Viscosity | 2.2 Pa · s | 7.3 Pa · s | not | 10 Pa · s (rotor | less than |
| | (rotor No. | (rotor No. | measured | No. 4) | 0.3 Pa · s |
| | 3) | 4) | | | |

(1) Aculyn 22
(2) Carbopol 1382
(3) Dehyton AB 30 from the company Cognis (aqueous solution comprising 30% of active substance)
(4) Proteol OAT (aqueous solution comprising 30% of active substance)
(5) Ascorbyl glucoside
(6) Empicol AL30/FL from the company Huntsman (aqueous solution comprising 30% of active substance)
(7) Texapon T42 from the company Cognis (aqeuous solution comprising 40% of active substance)
(8) Plantacare 818UP from the company Cognis (aqueous solution comprising 53% of active substance)
(9) Mydol 10 from the company KAO (aqueous solution comprising 40% of active substance)
(10) Merquat 2001 from the company Ondeo (aqueous solution comprising 20% of active substance)
(11) Calmiskin (Mentha piperita) from the company Silab (extract in aqueous solution)

The compositions of the examples described above have a pH of 5.8 to 6.3±0.3. They have a pearlescent, transparent and smooth appearance. Examples 1 to 4 present good foam initiation, give a fairly dense foam and have good cleansing powers. Example 5 has an insufficient viscosity, which causes a less satisfactory comfort during use, and the example shows that it is essential to have both the surfactant system and the thickening system in conformity with the invention in order to obtain a composition having the required foam viscosity and quality properties.

On a panel of 38 people who tested the composition of Example 1, 85% found that the foam was sufficient, that it was of sufficient quality and that the product was easy to rinse off, 82% found that the product was smooth and 94% found that the product was pleasant to use.

### EXAMPLE 6 ACCORDING TO THE INVENTION AND COMPARATIVE EXAMPLE 7

| | Example 6 | | |
|---|---|---|---|
| | according to | Comparative | Comparative |
| | the invention | Example 7 | Example 8 |
| Water | Qsp 100 | Qsp 100 | Qsp 100 |
| Glycerine | 4 | 4 | 4 |
| Methylparaben | 0.3 | 0.3 | 0.3 |
| Disodium EDTA | 0.25 | 0.25 | 0.25 |
| Sodium Lauroyl Oat Amino | 2 | 2 | 2 |
| Acids (1) | | | |
| Ammonium lauryl sulfate (2) | 8 | 8 | 8 |
| TEA Lauryl Sulfate (3) | 2 | 2 | 2 |
| Perfume | 0.2 | 0.2 | 0.2 |
| menthol | 0.05 | 0.05 | 0.05 |
| Decyl glucoside (4) | 2 | 2 | 2 |
| Cocoglucoside (5) | 2 | 2 | 2 |
| Cocobetaine (6) | 4.5 | 4.5 | 4.5 |
| Acrylates/Steareth-30 | 2.5 | 2.5 | 2.5 |
| Methacrylate | | | |
| copolymer (7) | | | |
| Acrylates/C10-30 Alkyl | 0.3 | — | — |
| acrylate Crosspolymer (8) | | | |
| Polyacrylamide/C13-14 | — | 0.3 | 0.3 |
| Isoparaffin/Laureth-7 (9) | | | |
| Triethanolamine | 0.2 | 0.2 | 0.5 |
| Appearance | Gelified | Liquid | Gelified |
| | composition | composition | composition |
| Viscosity at 25° C. | 3 Pa · s | 1.3 Pa · s | 3.1 Pa · s |
| (measured with | (30 poises) | (13 poises) | (31 poises) |
| Rheomat 180) | | | |

(1) Proteol OAT (aqueous solution comprising 30% of active substance)
(2) Empicol AL30/FL from the company Huntsman (aqueous solution comprising 30% of active substance)
(3) Texapon T42 from the company Cognis (aqeuous solution comprising 40% of active substance)
(4) Mydol 10 from the company KAO (aqueous solution comprising 40% of active substance)
(5) Plantacare 818UP from the company Cognis (aqueous solution comprising 53% of active substance)
(6) Dehyton AB 30 from the company Cognis (aqueous solution comprising 30% of active substance)
(7) Aculyn 22
(8) Carbopol 1382
(9) Sepigel 305 (from the company SEPPIC) (comprising 40% of active substance)

The example 6 according to the invention has a greater viscosity of the comparative example 7 that comprises only one anionic polymer comprising at least one hydrophobic chain, polymer which is associated with one anionic polymer without hydrophobic chain (Sepigel 305). Comparative example 7 is too liquid and has an insufficient viscosity, which causes a less satisfactory comfort during use.

Furthermore, the qualities of the composition of the invention are better than those of the comparative example 7, since the example 6 gives a better initiation of the foam and gives denser and more abundant foam, and it rinses off easier. At the contrary, the initiation of the foam with comparative example 7 is difficult and the obtained foam is neither dense nor abundant.

Comparative example 8 which comprises a greater amount of triethanolamine than example 7 has a viscosity similar to the viscosity of example 6 but its pH is 7 at the place of 6, 3 for examples 6 and 7, that is unfavorable for an use on the skin. Furthermore, its qualities of use are similar to those of example 7, thus very poorer than those of example 6 according to the invention.

The above written description of the invention provides a manner and process of making and using it such that any person skilled in this art is enabled to make and use the same, this enablement being provided in particular for the subject matter of the appended claims, which make up a part of the original description.

As used above, the phrases “selected from the group consisting of,” “chosen from,” and the like include mixtures of the specified materials.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A composition comprising, in a physiologically acceptable aqueous medium, (1) a surfactant system comprising at least one anionic surfactant and at least one amphoteric surfactant, and (2) a thickening system comprising at least two different anionic polymers each comprising at least one hydrophobic chain.

2. The composition according to claim 1, wherein it comprises 35 to 98% by weight of water relative to the total weight of the composition.

3. The composition according to claim 1, wherein said composition has a viscosity of 0.3 to 20 Pa.s.

4. The composition according to claim 1, wherein the quantity of surfactant system is 0.5 to 40% by weight relative to the total weight of the composition.

5. The composition according to claim 1, wherein the anionic surfactant is selected from the group consisting of the anionic derivatives of proteins of plant origin, amino acid derivatives, alkyl sulphates, alkyl ether sulphates, sulphonates, isethionates, taurates, sulphosuccinates, alkyl sulphoacetates, phosphates and alkyl phosphates, polypeptides, anionic derivatives of alkyl polyglucoside, and mixtures thereof.

6. The composition according to claim 1, wherein the anionic surfactant is selected from the group consisting of wheat, soybean, oat or silk protein hydrolysates comprising an alkyl chain having from 10 to 22 carbon atoms and their salts, and mixtures thereof.

7. The composition according to claim 1, wherein the anionic surfactant is selected from the group consisting of the sodium, potassium and/or ammonium salts of hydrolysates of oat protein comprising an alkyl chain having from 10 to 22 carbon atoms, and mixtures thereof.

8. The composition according to claim 1, wherein the amphoteric surfactant is selected from the group consisting of betaines, N-alkylamidobetaines and derivatives thereof, glycine derivatives, sultaines, alkyl polyaminocarboxylates, alkylamphoacetates and mixtures thereof.

9. The composition according to claim 1, comprising the amphoteric surfactant cocobetaine.

10. The composition according to claim 1, wherein the surfactant system further comprises one or more nonionic surfactants.

11. The composition according to claim 10, comprising at least one alkyl polyglucoside.

12. The composition according to claim 1, comprising an anionic polymer selected from the group consisting of copolymers of carboxylic acid with α,β-monoethylenic unsaturation, and mixtures thereof.

13. The composition according to claim 12, wherein the anionic polymer is selected from the group consisting of copolymers comprising at least one monomer of acrylates, methacrylates or itaconates of oxyethylenated C₁₂-C₃₀ fatty alcohol, copolymers comprising at least one monomer of acrylates or methacrylates of C₁₂-C₃₀ fatty acid, and mixtures thereof.

14. The composition according to claim 1, wherein the thickening system comprises at least one copolymer comprising at least one monomer of the acrylates, methacrylates or itaconates of oxyethylenated C₁₂-C₃₀ fatty alcohol type, and at least one copolymer comprising at least one monomer of the acrylates and methacrylates of C₁₂-C₃₀ fatty acid type.

15. The composition according to claim 1, wherein the thickening system comprises an acrylates/steareth-20 methacrylate copolymer and/or an acrylates/C10-C30 alkyl acrylates copolymer.

16. The composition according to claim 1, wherein the quantity of thickening system is 0.05 to 10% by weight relative to the total weight of the composition.

17. The composition according to claim 1, wherein said composition constitutes a cosmetic composition.

18. A method, comprising applying to the skin and/or the hair the composition of claim 1.

19. The method according to claim 18, wherein said method is
a method for cleansing the skin or hair, or for make-up-removal.

20. The method according to claim 18, wherein the composition according to claim 1 is applied to the skin in the presence of water to form a foam, and the foam formed and any dirt residues are removed by rinsing with water.
